(19) United States
(12) Patent Application Publication
(10) Pub. No.: US 2005/0106556 A1
(43) Pub. Date: May 19, 2005

(54) Bio-sensors

(76) Inventor: Solomzi Makohliso, Lausanne (CH)

Correspondence Address: HOWSON AND HOWSON;ONE SPRING HOUSE CORPORATION CENTER, BOX 457, 321 NORRISTOWN ROAD, SPRING HOUSE, PA, 19477, US

(21) Appl. No.: 10/503,331

(22) PCT Filed: Feb. 6, 2003
(86) PCT No.: PCT/EP03/02601

(30) Foreign Application Priority Data
Feb. 8, 2002 (GB) 0203053.4

(51) Int. Cl. C12M 1/34; G01F 1/64; G01N 27/26

(52) U.S. Cl. 435/4; 435/287.1; 205/777.5

(57) ABSTRACT

A device for monitoring ions secreted and/or taken up by a cell comprises at least one chamber (9) adapted to hold a culture medium (13) containing the cell (12) under test, a transducer surface (2) disposed within the or each said chamber (9) and arranged to be contactable by said culture medium when placed in said chamber, and an ion-detecting species (3) immobilised on said transducer surface (2), adapted to interact with an ion of interest and upon such interaction to transmit an electrical signal to said transducer surface (2). Electrical monitoring means (10), electrically connected to said transducer surface (2), are adapted to detect said electrical signal as an indication of the presence and/or concentration of the ion of interest. A method of use of the device is also disclosed.

The present invention relates to devices for monitoring cellular activity. In particular, but not exclusively, it relates to devices of use in the screening of candidate active substances for potentially useful pharmaceutical activity.

Cell-based assays play an important role in the screening and testing of potential drug candidates. Typically, once a particular cellular protein has been identified as being associated with a given disease, it may be designated as a drug target, and tests with a range of possible drugs then carried out to establish which (if any) of the candidate substance interacts with the protein of interest in a beneficial manner. Because of the huge number of potential active substances, the testing method needs to be both reliable and rapid, in order to achieve what is known in the industry as “high-throughput screening”.

The traditional method for screening potential active substances involves the use of candidate substances labelled with fluorescent markers; optical detection methods are then used to establish whether or not the candidate substance has bound to the protein of interest. A major problem associated with this approach is that the introduction of the fluorescent moiety into the cell under test may influence the interaction between the test substance and the target protein, leading to unreliable results or false “hits”, whose consequence ultimately is an escalation of the costs involved in the drug discovery process. There is therefore a requirement for methods that eliminate the need to introduce foreign molecules into the cell under test.

A number of different devices have been proposed in the past for carrying out cell-based assays by electrical means. Planar multi-electrode arrays (“MEAs”), for instance as described in U.S. Pat. No. 6,151,519 and U.S. Pat. No. 5,563,067, are designed to detect certain excitations of biological cells, but they suffer from a number of disadvantages. One major problem with MBA technology, when used with mammalian cells, is the difficulty of positioning the cell optimally and reproducibly over the microelectrode area. Another major problem is the poor quality of signal transfer at the cell/electrode interface.

Signal detection in MEA devices is accomplished via a capacitive coupling mechanism, based on the double layer capacitance arising at the electrolyte/electrode interface. Consequently, the signal recorded is a first derivative of the actual signal and only signals that result in a change of polarity, for example action potentials in neurons, can be detected (see, for example, Breckenridge L. J. et al “Advantages of using microfabricated extracellular electrodes for in vitro neuronal screening” J. Neurosci. Res. 42 (1995) 266-276). Therefore, virtually all work with MBAs has been confined to excitable cells, i.e. cells that can elicit action potentials (such as neurons, heart and muscle cells), which is only a subset of cells of experimental interest. Furthermore, it typically takes several days (or even more than a week) before such cells become excitable, and therefore measurable, under experimental conditions. These limitations provide significant practical drawbacks, and despite the fact that MEA technology has existed for some thirty years, it has not yet developed to the point of commercial viability.

Various electrochemical techniques for monitoring cell activity involving the use of fibre microelectrodes have been reviewed by Clark et al (“Electrochemistry in Neuronal Environments” pp 227-295 from Electroanalytical Chemistry, eds. Bard A. J., Rubinstein I, vol 20, 1998). Such methods require the tip of the microelectrode device to be brought as close as possible to the cell surface, typically within a few microns, with the aid of micromanipulators. While such devices can provide accurate data, they require skilled operation, and the technology is not well suited for commercial sensors.

It has also been reported that ion detection may be carried out using an ion-selective field effect transistor (ISFET), which is a field-effect transistor that is triggered by the presence of a given chemical species on its gate surface. However, the utility of such devices in applications involving cultured cells would be limited, since continuous exposure to the electrolytic culture medium would be deleterious to device performance.

A particularly important group of proteins which are the subject of much active research in the development of novel pharmaceutical treatments are the ion channels. These are cell membrane proteins that regulate the flow of physiologically important ions, such as Na⁺, Ca²⁺, K⁺ and Cl⁻, into and out of cells. All cells maintain homeostasis by the continuous exchange of inorganic ions such as these between intracellular and extracellular media, and any cellular activation is accompanied by a change in the extracellular concentration of one or more ions as the result of a change in the activity of associated ion-specific ion channels. Accordingly, changes in ion channel activity may be used as an indication of cellular activation, for example following the introduction of a pharmacologically active substance.

The conventional method for investigating ion channels is the patch clamp technique, in which a polished glass pipette is delicately brought into contact with the surface of the cell membrane and light suction applied through the pipette to provide a giga-ohm seal. Highly sensitive investigation, even of single ion channels, may thereby be achieved. However, on account of the technically demanding nature of this technique, it is not suitable for industrial applications such as high-throughput drug screening.

Various attempts have been made to develop improved patch clamp devices, with a view to achieving simplified and speedier operation. However, the common feature of all such techniques is the establishment of a giga-ohm seal. Not only is this technically difficult, but it also entails various practical drawbacks in the capabilities of the sensors. In particular, the process of inducing the cell to form a tight seal may trigger background activation, i.e. the activation of unintended cellular processes that can lead to false results. Furthermore, in order to study a single family or sub-family of ion channels, it is necessary to block the activity of other ion channels by means of pharmacological agents. This may alter the physiological behaviour of the cell, and also increases the cost and complexity of the procedure.

The shortcomings of the various available measurement techniques may be illustrated by the difficulties associated with monitoring Ca²⁺ channels. There are several cellular processes, which, upon cellular activation, lead to the opening of Ca²⁺ channels and the flow of Ca²⁺ ions from the exterior to the interior of the cell. Currently, all techniques for the rapid detection of Ca²⁺ ions measure only intracellular Ca²⁺ levels. However, there are a number of intracellular processes that culminate in the release of Ca²⁺ into the cell's cytoplasm, and since it is not possible to separate intracellular from extracellular contributions to total Ca²⁺ concentration, it is not possible to characterise cellular events related to Ca²⁺ influx using such methods. There is currently no available method for monitoring extracellular Ca²⁺ levels.

There is therefore a need for improved devices for carrying out ion-specific cell-based assays, which avoid the introduction into the cells of foreign molecules such as fluorescent markers and which enable assays of a wide range of different ionic fluxes to be carried out rapidly and easily.

The applicants have found that it is possible to construct an electrochemical sensor capable of detecting the presence and measuring the concentration of a variety of different ionic species commonly secreted and/or taken up by biological cells, by immobilising an ion-detecting species specific to the ion of interest on a transducer surface forming an electrode of the electrochemical sensor. Provided the ion-detecting species is capable of transmitting an electrical signal to the conducting surface upon interaction with the ion of interest, such signal may be detected by suitable electrical monitoring means as an indication of the presence and/or concentration of the ion of interest. Detection may be achieved by a number of electrical techniques, such as voltammetry, potentiometry, amperometry and impedance spectroscopy.

In one aspect, the invention provides a device for monitoring ions secreted and/or taken up by a cell, the device comprising: at least one chamber adapted to hold a culture medium containing the cell under test; a transducer surface disposed within the or each said chamber and arranged to be contactable by said culture medium when placed in said chamber; an ion-detecting species immobilised on said transducer surface, adapted to interact with an ion of interest and upon such interaction to transmit an electrical signal to said transducer surface; and electrical monitoring means electrically connected to said transducer surface, adapted to detect said electrical signal as an indication of the presence and/or concentration of the ion of interest.

Suitably, the ion-detecting species comprises at least one ionophore. Ionophores are lipophilic, electron-rich complexing agents that are capable of reversibly binding ions and transporting them across organic membranes by carrier translocation. These compounds posses excellent ion-selective recognition capabilities, and have found widespread utility as components of sensor devices for use in the direct measurement of ions such as H⁺, NH₄⁺, Li⁺, Na⁺, K⁺, Cs⁺, Mg²⁺, Ca²⁺, Cd²⁺, Sr²⁺, Ba²⁺, Rb⁺, Cu²⁺, Ag⁺, Pb²+, UO²⁻, Cl⁻, CO³⁻, NO²⁻, NO³⁻, ClO⁴⁻, NCS⁻; HCO₃₋, BF₄₋. The lipophilicity of ionophores causes a problem when used in conjunction with living cells, however, since they may easily cross the lipid-based cell membrane, resulting in cytotoxicity. Traditionally, ionophores are incorporated into polymeric membranes, for example of polyvinyl chloride (PVC), which avoids this problem. In the present invention, which provides for the use of ionophores in more simplified configurations without the encapsulation membrane, the problem of inadvertent cellular ingestion of the molecules is avoided by permanently immobilising the naked molecules on the sensor. This also prevents transverse mobility across the cell membrane.

Preferably, molecules containing suitable ionophore species are immobilised onto the conducting surface of the sensor by the “self-assembly” technique, initially described by Nuzzo R. G. et al., J. Am. Chem. Soc. 105 (1983) 4481. Such methods are thermodynamically driven and can be controlled to achieve optimal packing and orientation of the molecules, such that their ion-trapping domains are preferentially exposed and available for interaction. Self-assembly may readily be achieved in a manner known to the person skilled in the art by utilising an ionophore moiety attached to a hydrocarbon chain terminating in a thiol group. Provided the transducer surface of the sensor is composed of a suitable material, for example a metal such as gold, the ionophore-containing molecules become covalently bound to the surface, with the ionophore moieties separated from the surface by the length of the hydrocarbon chain. The length of the hydrocarbon chain may be varied according to need, typically a minimum hydrocarbon chain of eight carbons being required in order to obtain an ordered monolayer. Using surface engineering techniques such as surface plasmon resonance, which measure the average height and packing density of the monolayer, the packing density may be controlled to provide the required degree of chain movement which provides the best configuration for ion trapping, as determined with an electrochemical technique such as impedance spectroscopy.

Though in many cases cells under test may be brought into adequate proximity with the ion-detecting species without difficulty, for example as a result of simple sedimentation if (as is preferred) the transducer surface is disposed at the bottom of the chamber, for some applications it is preferred to facilitate signal transfer by providing means to promote cell adhesion. Thus, some embodiments of device according to the invention further comprise a cell-adhesion-promoting species immobilised on the transducer surface, adapted and positioned to interact with the cell under test and to hold the cell in the vicinity of the ion-detecting species.

The active moiety of the cell-adhesion-promoting species may be attached to the transducer surface, via a hydrocarbon chain and thiol linkage, in a similar manner to the ion-detecting species, using a self-assembly technique. Thus, the sensor device may be constructed to have a layer on the transducer surface that consists of regions bearing the ionophore moiety and others bearing the moiety for promoting cell adhesion. A number of different groups may be used as the cell-adhesion-promoting species. For example, certain synthetic peptide sequences derived from the extracellular matrix (for example, amino acid sequences RKRLQVQLSIRT, RGD, YIGSR, SIKVAV and KAFDITYVRLKF) are known to be effective in promoting cell adhesion. Other examples include cellulose nitrate, as well as amine-bearing, carboxylic-bearing and hydroxyl-bearing compounds.

The invention takes advantage of the reversible ion-binding capacity of molecules such as ionophores, in order to sense and measure the concentration of a given ionic species in the culture medium immediately surrounding a cell under test. Such an approach has significant advantages over the prior art patch clamp techniques, in particular that it is not necessary to establish a tight seal between the cell and the transducer, since the transducer is designed to have a high affinity for the ions under investigation. The elimination of the requirement for a tight seal also renders the technique applicable for use with any type of cultured cell, which is not the case with patch clamp methods. Furthermore, the invention makes it possible, through appropriate choice of ion-detecting species, to study the activity of specific families or sub-families of ion channel, without the need to block other ion channels.

Sensor devices according to the invention also have substantial advantages over prior art MEAs. In particular, the devices of the invention are significantly more sensitive, due to the fact that the ions of interest are effectively focussed onto the transducer surface by the action of the ion-detecting species. This enables the sensors to detect much smaller ionic fluxes than the large sinusoidal fluxes that are necessary for detection by MEAs. Consequently, the sensors may be used with a much wider range of cell types and to detect much more subtle changes than was possible previously.

One of the principal applications of devices according to the invention is in the screening of potentially pharmaceutically active substances. Because the pre-labelling of a target molecule is not necessary, the sensors may be used not only to test for molecules that interact with known targets, but may also be used to test the effects of “orphan proteins”, whose physiological targets are not known. The absence of labelling also removes the risk of false positives associated with labelling techniques.

The invention is of use in cell-based screening methods in general. For example, devices constructed according to the invention may comprise a multiplicity of said chambers and may be incorporated into multi-well microtiter plates, which represent the standard format for carrying out cell-based studies. By making this alteration, to incorporate a sensor in each well, a direct functional read-out may be acquired from each well.

Devices according to the invention may also incorporated into chip-based Microsystems for use in the field of genomic and proteomic analysis. Examples of existing microsystems include chip-based electrophoretic and polymerase chain reaction devices, as well as microfluidic devices to combine or link such devices to sources of reactants or to analysis solutions. Thus, the device of the invention may comprise at least one fluidic channel for transporting test fluids to and from the at least one chamber. In most cases, the origin of biomolecules of interest is cellular, and the invention therefore provides the possibility of a chip-based testing device that can be interfaced directly with the various existing micro-analytic devices.

The invention is hereinafter described in greater detail by way of example only, with reference to the accompanying drawings, in which:

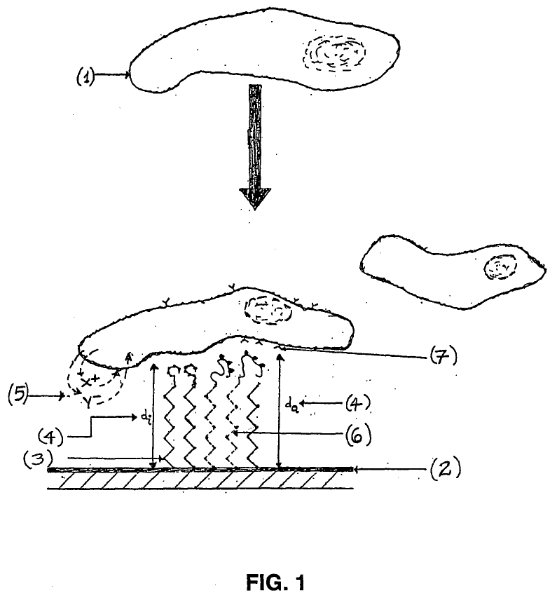

FIG. 1 is a diagrammatic vertical section through an embodiment of apparatus according to the invention, and also showing cells under test;

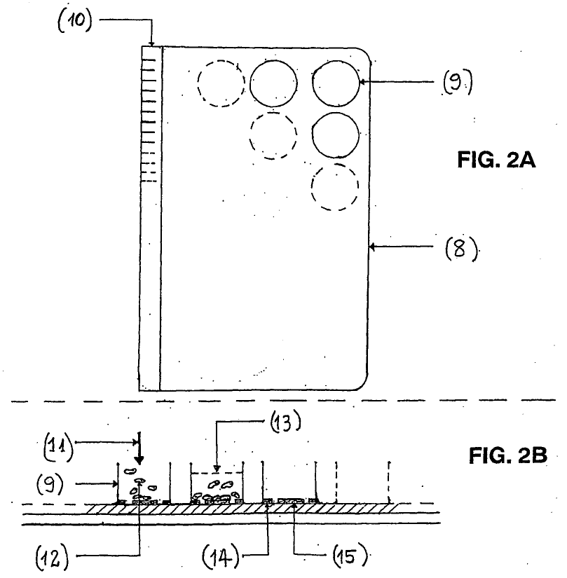

FIG. 2A is plan of an embodiment of apparatus according to the invention, configured in a multi-well format, with only some of the wells indicated;

FIG. 2B is a vertical section through the apparatus of FIG. 2A, also showing cells under test;

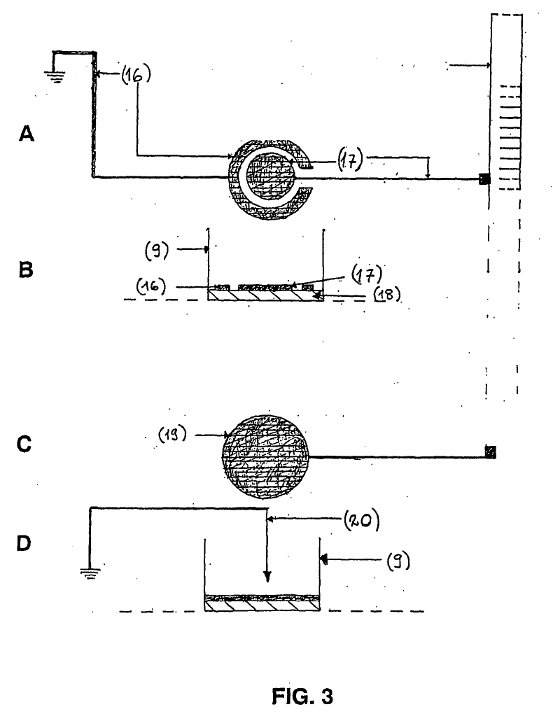

FIG. 3A is a diagram indicating the electrode configuration of an embodiment of apparatus according to the invention;

FIG. 3B is a vertical section through the electrode arrangement of FIG. 3A;

FIG. 3C is a diagram indicating the electrode configuration of an alternative embodiment of apparatus according to the invention; and

FIG. 3D is a vertical section through the electrode arrangement of FIG. 3C.

Depicted in FIG. 1 is a diagrammatic representation of the active area of a biochip comprising an active transducer surface (2) composed of gold. The gold layer is mounted on a substrate of a suitable material, for example an electrically insulating polymeric material such as polystyrene, polypropylene or polycarbonate. Onto the gold layer are adsorbed custom-synthesized ionophore-containing molecules (3), each consisting of an ionophore moiety, such as tridodecylamine (C₃₆H₇₅N), at one end, that has been grafted onto an n-alkyl thiol such as CH₃(CH₂)ₘSH (typically, m=8). The hybrid molecules are attached to the gold surface via the sulfhydryl/thiol group (SH). Also co-adsorbed onto the gold layer may be cell adhesion promoting molecules (6), shown in broken lines; these typically are custom-synthesised peptides (e.g. YIGSR or RGD) grafted onto an n-alkyl thiol, for example as indicated above (typically, m=18). The peptide-containing molecules are attached to the gold surface via their thiol groups in similar manner to the ionophore-containing molecules. Typically, the ionophore-containing molecules (for example in 1 to 2 mM solution) are first adsorbed on pristine gold for 2 to 3 hr, followed by the peptide-containing molecules (for example in a 0.1 mM solution) for another 2 hr.

The thickness of the molecular layer (4) depends on the overall lengths (respectively labelled dᵢ and dₐ) of the ionophore-containing and cell adhesion promoting molecules, both of which may readily be engineered by appropriate choice of hydrocarbon chain to suit the particular test conditions.

In use, a culture medium containing cells to be tested is placed in a chamber (not illustrated in FIG. 1), of which the gold transducer layer makes up the whole or part of the base. Cells in suspension sediment towards the transducer surface until they come into contact with the cell-adhesion-promoting molecules. Interaction between the latter and the appropriate cell surface receptors (7) then results in the cells becoming anchored to the biochip surface. The cell surface is thereby held in close proximity to the ionophore-containing molecules, which are thus in an optimal position to interact with the appropriate ions (e.g. K⁺, Na⁺, Ca²⁺) from the ion flux (5) passing into and out of the cell.

FIGS. 2A and 2B illustrate a multi-well device (8) suitable for the parallel testing of several different compounds or several different concentrations of the same compound. The preferred configuration of multi-well device is a microtiter plate format which incorporates a sensor at the bottom of each well. Also illustrated (10) is means for connecting external electronic signal acquisition and processing apparatus.

FIG. 2B illustrates the micro-wells (9) in section, with reference numeral 11 representing the insertion of cells (12) in a culture medium (13), typically a physiologically balanced electrolyte, which is normally carried out using a culture pipette. The majority of cells sediment to the bottom of the well (as indicated in the well illustrated second from left), and are thus brought into contact with the biosensor interface. The chamber format allows the possibility for a given volume of electrolyte to cover the cell layer at the bottom of the well at all times throughout the test procedure. Also illustrated in FIG. 2B is one of the preferred configurations of electrode, consisting of a common reference/ground line (14) and an active region (15).

Preferred electrode configurations are shown in more detail in FIG. 3. In FIGS. 3A and 3B, the common reference line (16) is integrated into the baseplate of the device, along with the active region (17). FIG. 3B corresponds to one of the micro-wells shown on FIG. 2.

FIGS. 3C and 3D illustrate alternative arrangement, in which the bottom of the micro-well is covered with the active electrode (19) and the reference electrode (20) is introduced into the chamber from above.

All forms of the verb “to comprise” used in this specification have the meaning “to consist of or include”.

1. A device for monitoring ions secreted and/or taken up by a cell, the device comprising:
at least one chamber adapted to hold a culture medium containing the cell under test;
a transducer surface disposed within the or each said chamber and arranged to be contactable by said culture medium when placed in said chamber;
an ion-detecting species immobilised on said transducer surface, adapted to interact with an ion of interest and upon such interaction to transmit an electrical signal to said transducer surface; and
electrical monitoring means electrically connected to said transducer surface, adapted to detect said electrical signal as an indication of the presence and/or concentration of the ion of interest.

2. A device according to claim 1, wherein said ion-detecting species comprises at least one ionophore.

3-10. (cancel)

11. A device according to claim 2, further comprising a cell-adhesion-promoting species immobilised on said transducer surface, adapted and positioned to interact with the cell under test and to hold said cell in the vicinity of said ion-detecting species.

12. A device according to claim 11, wherein the transducer surface is composed of a metal, carbon or a conductive polymer.

13. A device according to claim 12, wherein the conducting surface is composed of gold.

14. A device according to claim 13, comprising a multiplicity of said chambers.

15. A device according to claim 14, comprising at least one fluidic channel for transporting test fluids to and from the at least one chamber.

16. A device according to claim 1, further comprising a cell-adhesion-promoting species immobilised on said transducer surface, adapted and positioned to interact with the cell under test and to hold said cell in the vicinity of said ion-detecting species.

17. A device according to claim 1, wherein the transducer surface is composed of a metal, carbon or a conductive polymer.

18. A device according to claim 17, wherein the conducting surface is composed of gold.

19. A device according to claim 1, comprising a multiplicity of said chambers.

20. A device according to claim 1, comprising at least one fluidic channel for transporting test fluids to and from the at least one chamber.

21. A method of detecting the presence and/or concentration of an ionic species secreted by a cell under test, comprising the steps of:
placing a sample of a culture medium containing the cell under test in a chamber of a device having: a chamber adapted to hold a culture medium containing the cell under test; a transducer surface disposed within said chamber and arranged to be contactable by said culture medium when placed in said chamber; an ion-detecting species immobilised on said transducer surface, adapted to interact with an ion of interest and upon such interaction to transmit an electrical signal to said transducer surface; and electrical monitoring means electrically connected to said transducer surface, adapted to detect said electrical signal as an indication of at least one of the presence and concentration of the ion of interest; and
monitoring said electrical monitoring means for a signal indicative of the presence of the ion of interest.

22. A method according to claim 21, further comprising the step of providing a stimulus to the cell under test, and wherein the electrical monitoring means is monitored for a signal indicative of the ion of interest both before and after the application of said stimulus.

23. A method for establishing the effect of a candidate active substance on a cell, comprising the steps of:
placing a sample of a culture medium containing the cell under test in a chamber of a device having: a chamber adapted to hold a culture medium containing the cell under test; a transducer surface disposed within said chamber and arranged to be contactable by said culture medium when placed in said chamber; an ion-detecting species immobilised on said transducer surface, adapted to interact with an ion of interest and upon such interaction to transmit an electrical signal to said transducer surface; and electrical monitoring means electrically connected to said transducer surface, adapted to detect said electrical signal as an indication of at least one of the presence and concentration of the ion of interest;
monitoring said electrical monitoring means for a signal indicative of the presence of the ion of interest; and
adding said candidate substance to the culture medium.
